(19) United States
(12) Patent Application Publication
(10) Pub. No.: US 2025/0248906 A1
(43) Pub. Date: Aug. 7, 2025

(54) CREAM MAKER WAX

(71) Applicant: AMIRHOSSEIN AREFIKIA, ISFAHAN (IR)

(72) Inventor: AMIRHOSSEIN AREFIKIA, ISFAHAN (IR)

(21) Appl. No.: 19/008,538

(22) Filed: Jan. 2, 2025

Related U.S. Application Data
(63) Continuation-in-part of application No. PCT/IB2023/053401, filed on Apr. 4, 2023.

(51) Int. Cl. A61K 8/06; A61K 8/34; A61K 8/37; A61K 8/49; A61K 8/60; A61K 8/92; A61Q 19/00

(52) U.S. Cl. CPC A61K 8/06; A61K 8/342; A61K 8/37; A61K 8/375; A61K 8/4973; A61K 8/60; A61K 8/922; A61Q 19/00; A61K 2800/10

(57) ABSTRACT

In accordance with some embodiments herein, a method for producing a wax product is provided. The method includes forming a first mixture by mixing Poly ethylene glycol 40 Hydrogenated Castor Oil (PEG 40 HCO) and Isopropyl myristate (C17H34O2) using a first reactor, forming a second mixture based upon mixing Polysorbate 80 (C64H124O26) and Sorbitan monooleate (C24H44O6) using a second reactor, forming a third mixture based upon mixing Glycerin monostearate (C21H42O4) and Stearyl Alcohol (C18H38O) using a third reactor, and combining the first mixture, the second mixture and the third mixture to produce the wax product.

### RELATED APPLICATION

This application claims priority to and is a continuation-in-part of International Application Number PCT/IB2023/053401, filed on Apr. 4, 2023, entitled “CREAM MAKER WAX”, which is incorporated by reference herein in its entirety.

### TECHNICAL FIELD

The present application relates to the technical field of health and cosmetic production, for example, to a base cream and a substance and a process for making the base cream which may be used in cosmetic products, pharmaceutical products and/or industrial products.

### BACKGROUND

A cream is a type of emulsion, made of a water phase and an oil phase. An emulsion is a milky moisturizer with a gel-like consistency. Emulsions are water-based, and somehow are used as a lighter type of moisturizing creams. This makes emulsions a lightweight alternative to traditional creams and face oils or creams, which can leave a greasy, weighed down feeling. Oil or fat does not readily dissolve or disperse in water, so in order to achieve this, a dispersing agent called an emulsifier is added to the mixture. Based on the HLB number of emulsifiers, they are divided into two categories: water-loving (polar) and oil-loving (nonpolar or hydrophobic) types, or correspondingly Water-in-Oil (W/O) and/or Oil-in-Water (O/W).

The main use of emulsifiers is that it dissolves compounds such as water and oil which are immiscible and do not combine with each other even by stirring. An emulsifier is used to prevent the separation of the components and to stabilize fine particles of two liquids.

### SUMMARY

This summary is provided to introduce a selection of concepts in a simplified form that are further described below in the detailed description. This summary is not intended to identify key factors or essential features of the claimed subject matter, nor is it intended to be used to limit the scope of the claimed subject matter.

In some embodiments, a method for producing a wax product is provided. The method includes forming a first mixture by mixing Poly ethylene glycol 40 Hydrogenated Castor Oil (PEG 40 HCO) and Isopropyl myristate (C17H34O2) using a first reactor, forming a second mixture based upon mixing Polysorbate 80 (C64H124O26) and Sorbitan monooleate (C24H44O6) using a second reactor, forming a third mixture based upon mixing Glycerin monostearate (C21H42O4) and Stearyl Alcohol (C18H38O) using a third reactor, and combining the first mixture, the second mixture and the third mixture to produce the wax product.

### BRIEF DESCRIPTION OF DRAWINGS

Aspects of the present disclosure are best understood from the following detailed description when read with the accompanying figures. It is noted that, in accordance with the standard practice in the industry, various features are not drawn to scale. In fact, the dimensions of the various features may be arbitrarily increased or reduced for clarity of discussion.

FIG. 1A-1D illustrate an example scenario in which a wax product is produced, in accordance to some embodiments.

FIG. 2A-2D illustrate an example scenario in which a wax product is produced, in accordance to some embodiments.

FIG. 3A-3C illustrate an example scenario in which a wax product is produced, in accordance to some embodiments.

FIG. 4A-4D illustrate an example scenario in which one or more mixtures are combined, in accordance to some embodiments.

FIG. 5A-5C illustrate an example scenario of producing a wax product, in accordance to some embodiments.

FIG. 6A-6F illustrate an example scenario of making one or more emulsions utilizing the wax product 550, in accordance to some embodiments.

FIG. 7 is a flow diagram illustrating a method for producing a wax product, in accordance to some embodiments.

### DETAILED DESCRIPTION

Subject matter will now be described more fully hereinafter with reference to the accompanying drawings, which form a part hereof, and which show, by way of illustration, specific example embodiments. This description is not intended as an extensive or detailed discussion of known concepts. Details that are known generally to those of ordinary skill in the relevant art may have been omitted, or may be handled in summary fashion.

The following subject matter may be embodied in a variety of different forms, such as methods, devices, components, and/or systems. Accordingly, this subject matter is not intended to be construed as limited to any example embodiments set forth herein. Rather, example embodiments are provided merely to be illustrative.

The present disclosure provides a wax product (e.g. an emulsion). In some embodiments, the wax product may comprise one or more mixtures. In some examples, the one or more mixtures may comprise one or more components. In some examples, the one or more mixtures may be produced utilizing one or more reactors. In some examples, the one or more reactors may control a temperature of the one or more mixtures to facilitate the chemical reactions occurring within the one or more mixtures. Alternatively and/or additionally, a liquid and/or an oil may be added to the wax product to produce at least one of a cosmetic product, a pharmaceutical product, or an industrial product.

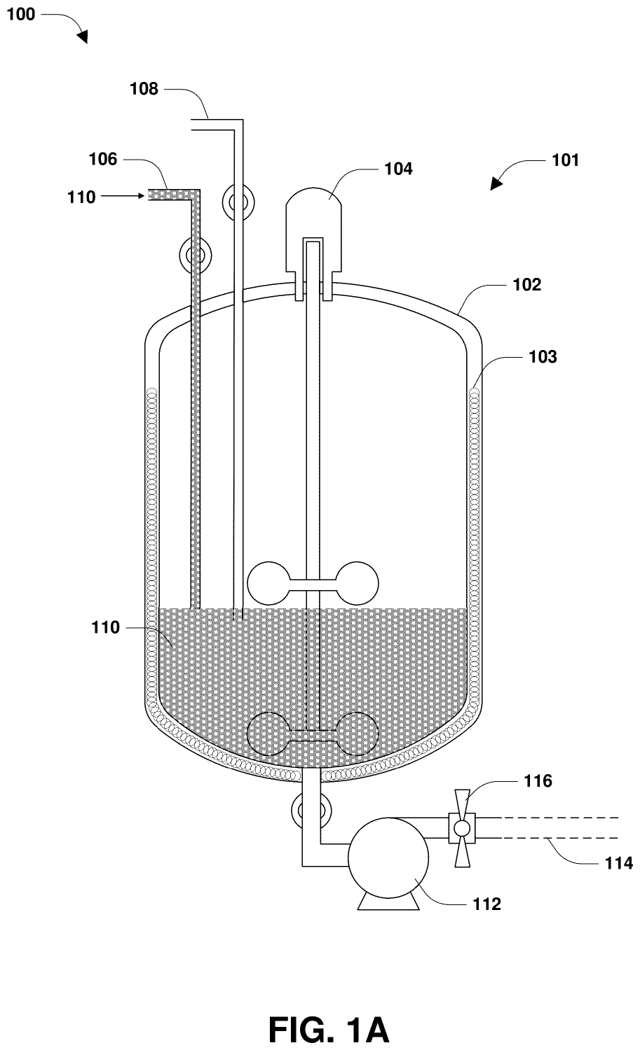

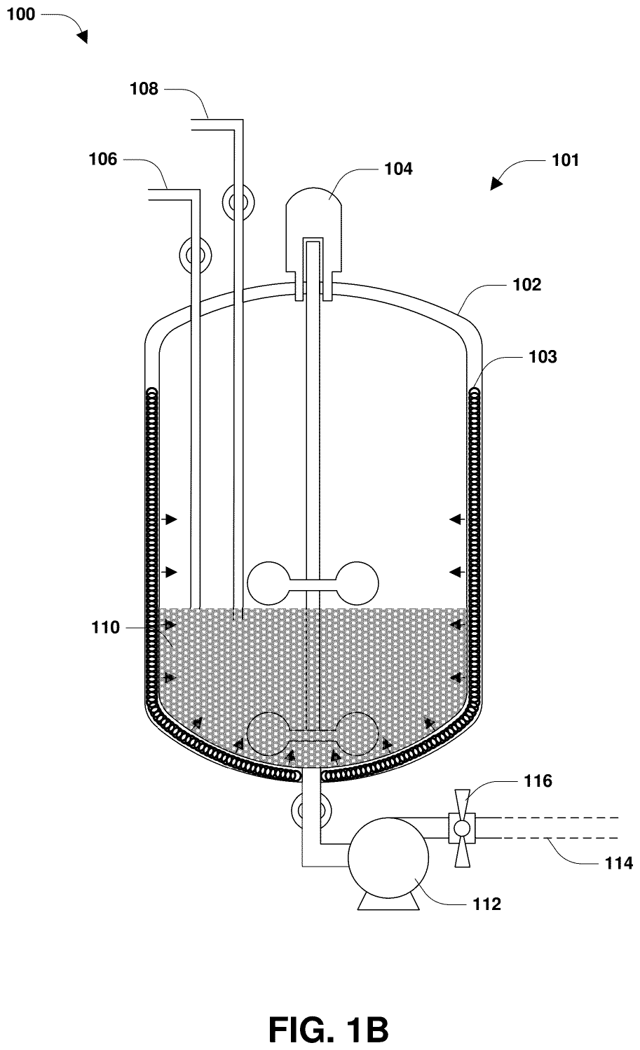

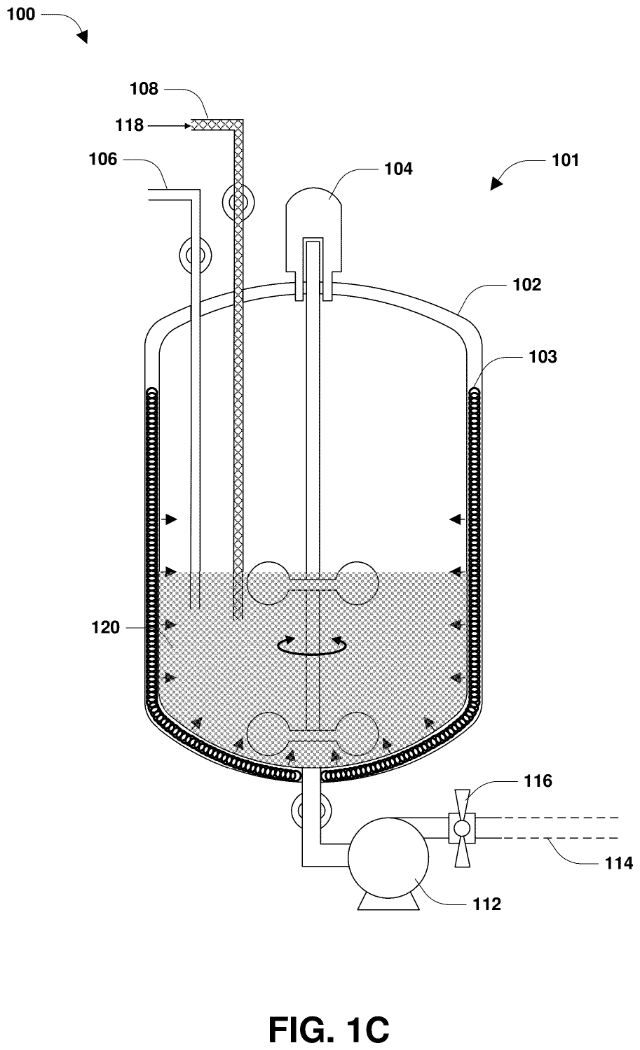

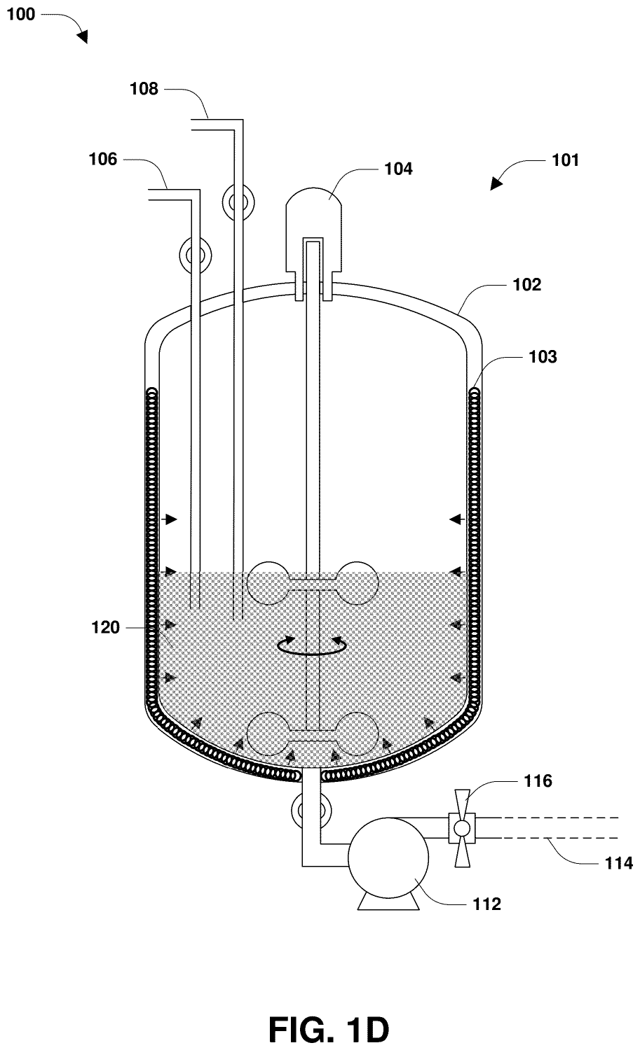

FIG. 1A-1D illustrate an example scenario 100 in which a wax product is produced, in accordance to some embodiments. In some examples, a first reactor 101 (e.g., bioreactor) is utilized to produce the wax product. The first reactor 101 may comprise at least one of (i) a first chamber 102, (ii) a first mixer 104 (e.g., to mix one or more components), (iii) one or more inlets (e.g., an inlet 106, an inlet 108, etc.), (iv) a first heater 103 (e.g., to control and provide heat for the one or more components), or (v) a first pump 112. In some examples, the first chamber 102 may comprise one or more materials. The one or more materials may comprise at least one of metals, glasses, ceramics, polymers, carbon, or composites. In FIG. 1A, a first component 110 may enter the first chamber 102 via the inlet 106. In some examples, the first component 110 may be a Poly Ethylene Glycol 40 Hydrogenated Castor Oil (PEG 40 HCO). In some examples, a ratio of the first component 110 (e.g., the PEG 40 HCO) to the wax product by weight is between about 0.1:1 to about 0.15:1. In some examples, the first component 110 may be a liquid with a first viscosity (e.g., high viscosity).

In some examples, a first temperature of the first component 110 is controlled using the first heater 103 (e.g., the first heater 103 provides heat to the first component 110 to increase the first temperature of the first component 110 or ceases providing heat to the first component 110 to maintain or decrease the first temperature of the first component 110). In FIG. 1B, the first temperature of the first heater 103 may be controlled (using the first heater 103, for example) to be one or more temperatures within a first temperature range. In some examples, the first temperature range ranges from between about 30 degrees Celsius to about 80 degrees Celsius. In some examples, the first temperature range ranges from about 50 degrees Celsius to about 70 degrees Celsius (e.g., 65 degrees Celsius). In some examples, based upon maintaining the first temperature, a viscosity (e.g., relatively high viscosity) of the first component 110 may decrease and the first component 110 may become a diluted liquid oil. In FIG. 1C, a second component 118 may enter (e.g., added slowly) the first chamber 102 via the inlet 108. In some examples, the second component 118 may be an isopropyl myristate (C17H34O2). In some examples, a ratio of the second component 118 (e.g., the isopropyl myristate) to the wax product by weight is between about 0.005:1 to about 0.02:1. In some examples, a first mixer 104 is used to form a first mixture 120 based upon combining the first component 110 with the second component 118. In some examples, the first mixer 104 may mix (e.g., stir) the first mixture 120 with a first stirring speed in range of about 10 revolutions per minute (rpm) to about 250 rpm. In some examples, the first mixer 104 may mix the first mixture 120 with the first stirring speed in range of about 50 rpm to about 100 rpm. In FIG. 1D, the first temperature of the first component 110 may be decreased (using the first heater 103, for example) by a first temperature decrease. In some examples, the first temperature decrease may be between about 1 degree Celsius to about 15 degrees Celsius. In some examples, the first temperature decrease may be between about 4 degrees Celsius to about 10 degrees Celsius. In some examples, upon applying the first temperature and/or the first stirring speed, the first component 110 may dissolve in the second component 118 and form the first mixture 120. In some examples, the first mixture 120 may be a homogeneous mixture (e.g., a uniformed mixture). In some examples, the first mixer 104 may continue mixing the first mixture 120 at the first temperature for a first time period. In some examples, the first time period may be in a range of about 5 minutes to about 40 minutes. In some examples, the first time period may be in a range of about 10 minutes to about 30 minutes. In some examples, the first pump 112 may allow transferring the first mixture 120 utilizing a first valve 116 and/or a first outlet 114.

In some examples, the first component 110 (e.g., PEG 40 HCO) may be an emulsifier and/or a surfactant which is produced from a reaction of 40 moles of ethylene oxide with 1 mole of hydrogenated castor oil. In some examples, the first component 110 may be used as at least one of a softener, a hydrator, an emulsifier, or a moisturizing agent. In some examples, the first component 110 may be mixed with a surfactant and/or an emulsifier (e.g., polysorbate 80) to form a mixture. In some examples, the mixture may increase emulsifying properties of a final product (e.g., an emulsion) and create a pure white emulsion. In some examples, the first component 110 may be used to (i) mix immiscible compounds, (ii) improve the spreading of cosmetics and health products on a skin (e.g., a human skin), (iii) improve the effect on the skin and creating softness in the skin. In some examples, the first component 110 may be used in at least one of a hydrophilic emulsifier, an oil solubilizer, or a stabilizer of additives. In some examples, the first component 110 may be used as an effective solvent for isopropyl myristate. In some examples, the first component 110 may be used to dissolve one or more vitamins.

In some examples, the second component 118 (e.g., isopropyl myristate) may be an ester of isopropyl alcohol and/or a myristic acid with an HLB value of about 11.5. In some examples, the second component 118 may be a moisturizer with polar properties that is used in topical cosmetic products for skin treatments. In some examples, the second component 118 may be used largely as a penetration enhancer, softener and retains moisture. In some examples, the second component 118 creates a silky feeling instead of being greasy, and improves the texture of the products (e.g., products which have a high oil content. In some examples, the second component 118 may comprise emulsifying properties and helps emulsifiers and/or surfactants to perform better. In some examples, the second component 118 provides softening properties, greater penetration, silky feeling and/or improved texture to one or more final products (e.g., cosmetic products, pharmaceutical products, industrial products, etc.).

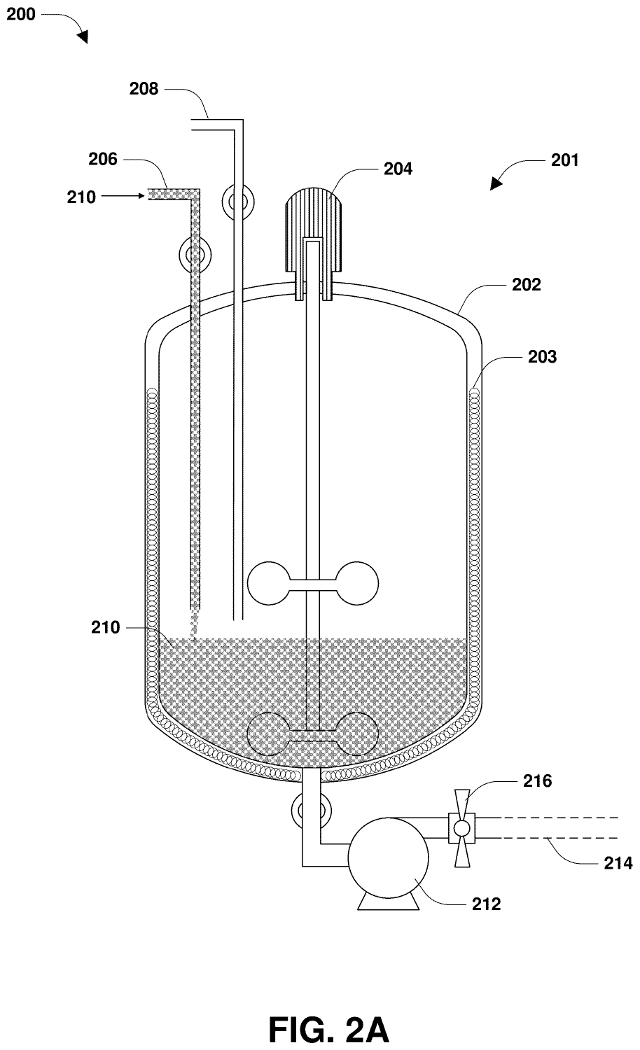

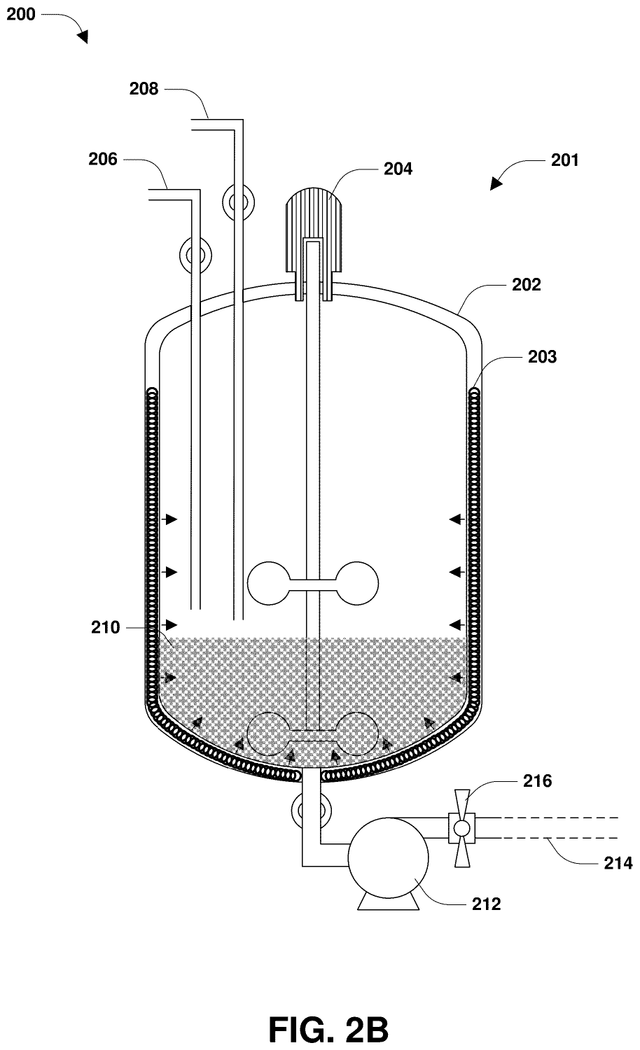

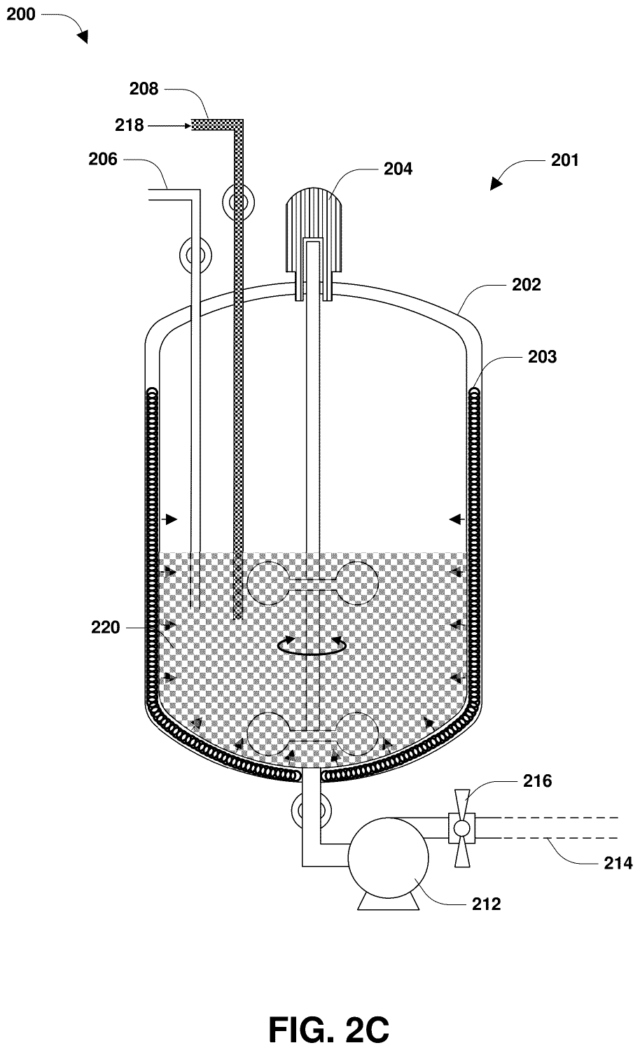

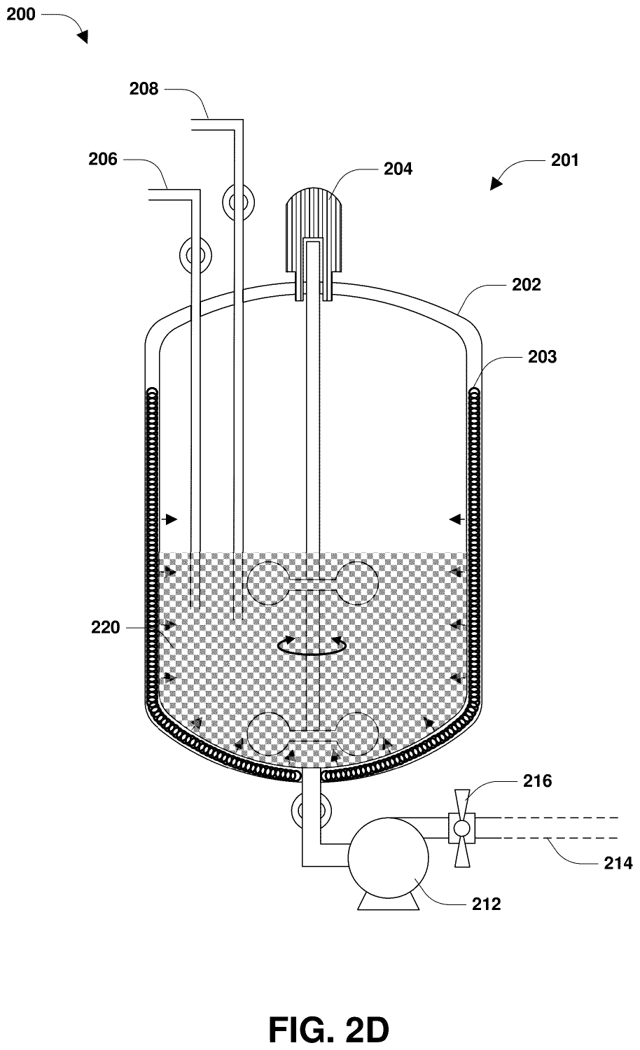

FIG. 2A-2D illustrate an example scenario 200 in which a wax product is produced, in accordance to some embodiments. In some examples, a second reactor 201 (e.g., bioreactor) is utilized to produce the wax product. The second reactor 201 may comprise at least one of (i) a second chamber 202, (ii) a second mixer 204 (e.g., to mix one or more components), (iii) one or more inlets (e.g., an inlet 206, an inlet 208, etc.), (iv) a second heater 203 (e.g., to control and provide heat for the one or more components), or (v) a second pump 212. In some examples, the second chamber 202 may comprise one or more materials. The one or more materials may comprise at least one of metals, glasses, ceramics, polymers, carbon, or composites. In FIG. 2A, a third component 210 may enter the second chamber 202 via the inlet 206. In some examples, the third component 210 may be a polysorbate 80 (C64H124O26). In some examples, a ratio of the third component 210 (e.g., the polysorbate 80) to the wax product by weight is between about 0.08:1 to about 0.13:1. In some examples, the third component 210 may be a liquid.

In some examples, a second temperature of the third component 210 is controlled using the second heater 203 (e.g., the second heater 203 provides heat to the second component 210 to increase the second temperature of the third component 210 or ceases providing heat to the third component 210 to maintain or decrease the second temperature of the third component 210). In FIG. 2B, the second temperature of the second heater 203 may be controlled (using the second heater 203, for example) to be one or more temperatures within a second temperature range. In some examples, the second temperature range ranges from between about 30 degrees Celsius to about 80 degrees Celsius. In some examples, the second temperature range ranges from about 50 degrees Celsius to about 70 degrees Celsius (e.g., 65 degrees Celsius). In some examples, based upon maintaining the second temperature, a viscosity (e.g., relatively high viscosity) of the third component 210 may decrease and the third component 210 may become a diluted liquid oil. In FIG. 2C, a fourth component 218 may enter (e.g., added slowly) the second chamber 202 via the inlet 208. In some examples, the fourth component 218 may be a sorbitan monooleate (C24H44O6). In some examples, a ratio of the fourth component 218 (e.g., sorbitan monooleate) to the wax product by weight is between about 0.05:1 to about 0.12:1. In some examples, the second mixer 204 is used to form a second mixture 220 based upon combining the third component 210 with the fourth component 218. In some examples, the second mixer 204 may mix the second mixture 220 with a second stirring speed in range of about 10 rpm to about 250 rpm. In some examples, the second mixer 204 may mix the second mixture 220 with the second stirring speed in range of about 50 rpm to about 100 rpm. In FIG. 2D, the second heater 203 may decrease the second temperature by a range of about 1 degree Celsius to about 15 degrees Celsius. In some examples, the second heater 203 may decrease the second temperature by a range of about 4 degrees Celsius to about 10 degrees Celsius. In some examples, upon applying the second temperature and/or the second stirring speed, the third component 210 may dissolve in the fourth component 218 and form the second mixture 220. In some examples, the second mixture 220 may be a homogeneous mixture (e.g., a uniformed mixture). In some examples, the second mixer 204 may continue mixing the second mixture 220 at the second temperature for a second time period. In some examples, the second time period may be in a range of about 5 minutes to about 40 minutes. In some examples, the second time period may be in a range of about 10 minutes to about 30 minutes. In some examples, the second pump 212 may allow transferring the second mixture 220 utilizing a second valve 216 and/or a second outlet 214.

In some examples, the third component 210 (e.g., polysorbate 80) may be produced form polyethoxylated sorbitan and/or oleic acid. In some examples, the HLB value of the third component 210 may be about 15 and based upon the HLB value, the third component 210 is highly hydrophilicity. In some examples, the third component 210 may be used as an emulsifier and/or a surfactant (e.g., in the second mixture 220), in which the third component 210 may cause a liquid (e.g., water) and/or an oil dissolve. In some examples, the third component 210 is compatible with products with high water percentages and may form a final composition.

In some examples, the fourth component 218 (e.g., sorbitan monooleate) may be an emulsifier and/or surfactant. In some examples, based upon using the fourth component 218 the properties of emulsifying and mixing (e.g., dissolvement) ratio of water and oil may be increased and as a result the stability of the second mixture 220 may be improved. Sorbitan monooleate has an HLB equal to 4.3 and its low HLB indicates its lipophilic (oil-loving) property, which makes the composition able to absorb more oil and the composition can resist high oil percentages and can absorb high oil percentages easily. Since it has a low HLB, it is also used as an HLB reducer, and also it has emulsifier, surfactant, solvent, stabilizer, softening, and anti-inflammatory properties.

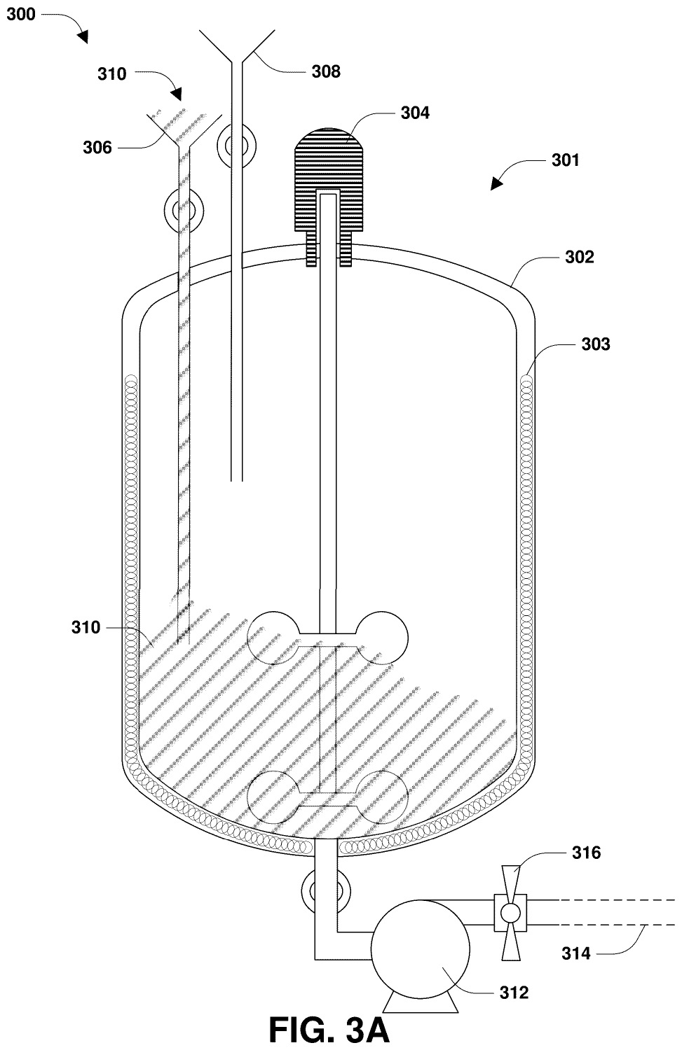

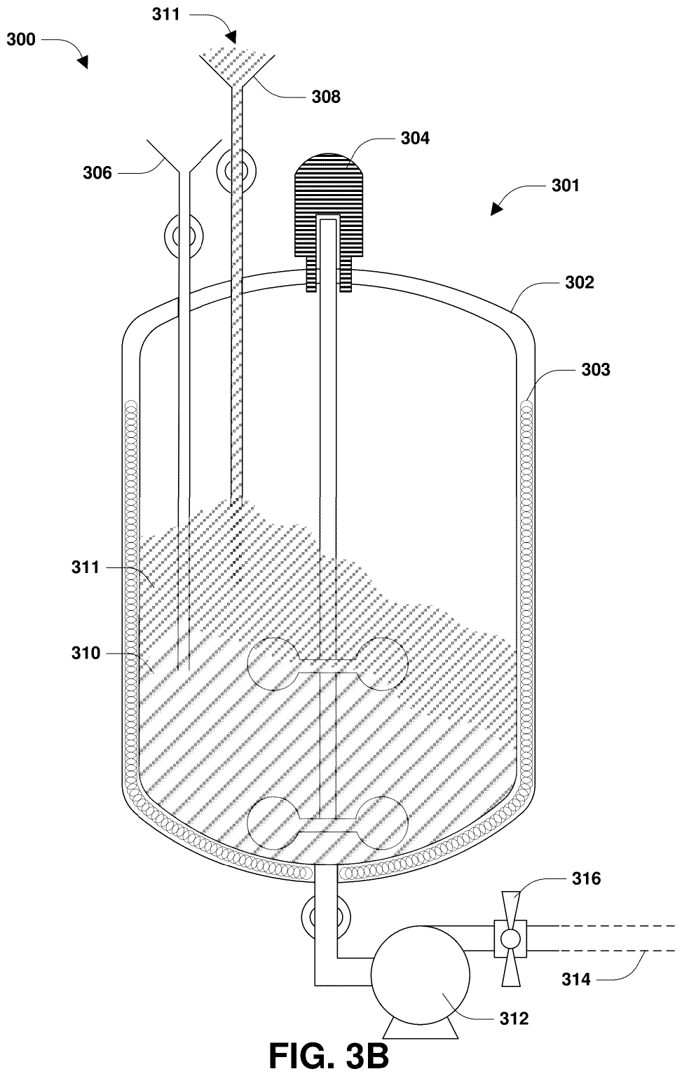

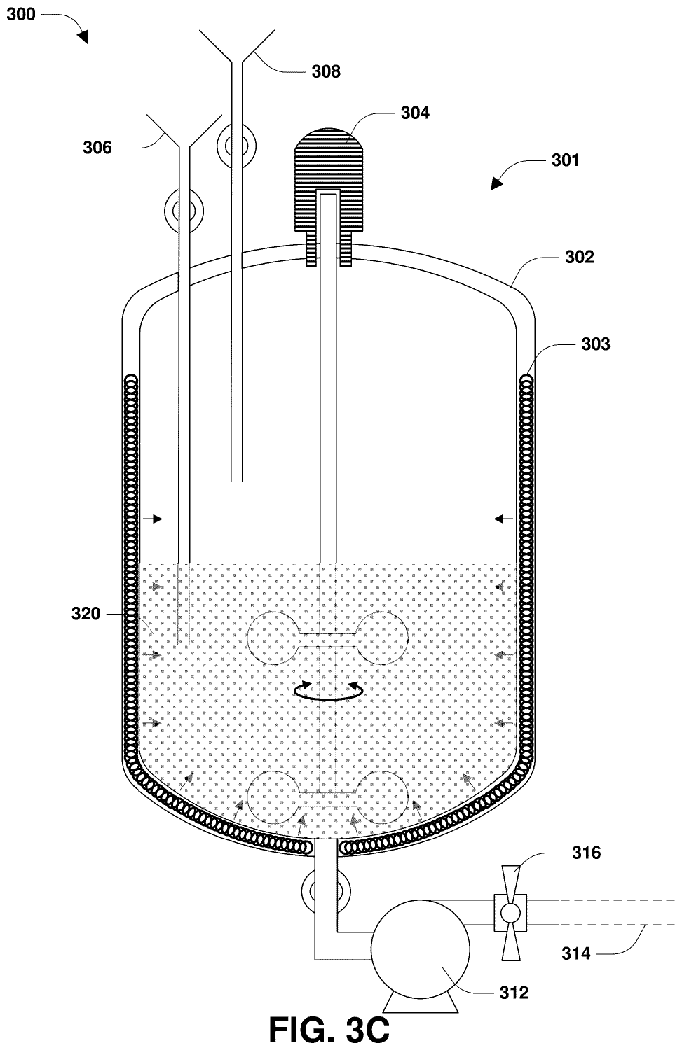

FIG. 3A-3C illustrate an example scenario 300 in which a wax product is produced, in accordance to some embodiments. In some examples, a third reactor 301 (e.g., bioreactor) is utilized to produce the wax product. The third reactor 301 may comprise at least one of (i) a third chamber 302, (ii) a third mixer 304 (e.g., to mix one or more components), (iii) one or more inlets (e.g., an inlet 306, an inlet 308, etc.), (iv) a third heater 303 (e.g., to control and provide heat for the one or more components), or (v) a third pump 312. In some examples, the third chamber 302 may comprise one or more materials. The one or more materials may comprise at least one of metals, glasses, ceramics, polymers, carbon, or composites. In FIG. 3A, a fifth component 310 may enter the third chamber 302 via the inlet 306. In some examples, the fifth component 310 may be a glycerin monostearate (C21H42O4). In some examples, a ratio of the fifth component 310 (e.g., the glycerin monostearate) to the wax product by weight is between about 0.2:1 to about 0.25:1. In some examples, the fifth component 310 may be a solid and based upon heating, the fifth component 310 may turn into a liquid.

In some examples, a third temperature of the fifth component 310 is controlled using the third heater 303 (e.g., the third heater 303 provides heat to the fifth component 310 to increase the third temperature of the fifth component 310 or ceases providing heat to the fifth component 310 to maintain or decrease the third temperature of the fifth component 310). In FIG. 3B, the third temperature of the third heater 303 may be controlled (using the third heater 303, for example) to be one or more temperatures within a third temperature range. In some examples, the third temperature range ranges from between about 50 degrees Celsius to about 180 degrees Celsius. In some examples, the third temperature range ranges from between about 65 degrees Celsius to about 100 degrees Celsius (e.g., 65 degrees Celsius). In some examples, based upon maintaining the third temperature, high viscosity of the fifth component 310 may decrease and the fifth component 310 may become a diluted liquid oil. In FIG. 1C, a sixth component 311 may enter (e.g., added slowly) the third chamber 302 via the inlet 308. In some examples, the sixth component 311 may be a stearyl alcohol (C18H38O). In some examples, a ratio of the sixth component 311 (e.g., the stearyl alcohol) to the wax product by weight is between about 0.3:1 to about 0.4:1. In some examples, the third mixer 304 is used to form a third mixture 320 based upon combining the fifth component 310 with the sixth component 311. In some examples, the third mixer 304 may mix (e.g., stir) the third mixture 320 with a third stirring speed in range of about 10 rpm to about 250 rpm. In some examples, the third mixer 304 may mix the third mixture 320 with the third stirring speed in range of about 50 rpm to about 100 rpm. In FIG. 1D, the third heater 303 may decrease the third temperature by a range of about 1 degree Celsius to about 15 degrees Celsius. In some examples, the third heater 303 may decrease the third temperature by a range of about 4 degrees Celsius to about 10 degrees Celsius. In some examples, upon applying the third temperature and/or the third stirring speed, the fifth component 310 may dissolve in the sixth component 311 and form the third mixture 320. In some examples, the third mixture 320 may be a homogeneous mixture (e.g., a uniformed mixture). In some examples, the third mixer 304 may continue mixing the third mixture 320 at the third temperature for a third time period. In some examples, the third time period may be in a range of about 5 minutes to about 40 minutes. In some examples, the third time period may be in a range of about 10 minutes to about 30 minutes. In some examples, the third pump 312 may allow transferring the third mixture 320 utilizing a third valve 316 and/or a third outlet 314.

In some examples, the fifth component 310 (e.g., glycerol monostearate) may be an emulsifier (e.g., emulsifying agent) and/or a thickening agent for an oil with moisture absorbing property. In some examples, the HLB value of the fifth component 310 may be about 3.5. In some examples, the fifth component 310 may enhance and/or control the release of medicinal compounds in one or more pharmaceutical products and/or in cosmetic products. In some examples, based upon using the fifth component 310 on a skin (e.g., a human skin), the skin may appear smoother. In some examples, the fifth component 310 may create a barrier on the skin and prevents the loss of water content of the skin (e.g., moisture the skin), furthermore the fifth component 310 may perform as a lubricant on the surface of the skin. In some examples, the fifth component 310 may help to form a stable emulsion and/or be used as a stabilizer in a composition (e.g., the wax product). In some examples, the fifth component 310 may cause the lack of dual phase (bi phasic) of the composition over time and/or increases the strength of stability of the composition as well as the cohesion (e.g., viscosity) of the composition.

In some examples, the sixth component 311 (e.g., the stearyl alcohol) may be an organic compound which is classified as a saturated fatty alcohol with the formula CH3(CH2)16CH2OH which may be used as an ingredient in one or more products such as lubricants, resins, perfumes, and one or more cosmetic products. In some examples, the sixth component 311 may comprise very high thickening and viscosifying properties which may increase cohesion in one or more products. In some examples, the sixth component 311 may comprise skin softening properties, hair softening properties, and emulsifying properties. In some examples, the sixth component 311 may be used as a thickener in creams, ointments, and lotions, furthermore the sixth component 311 may be used as a covering and softener for skin and hair. In some examples, the sixth component 311 may comprise thickening and/or viscous properties and be used as an emulsion stabilizer.

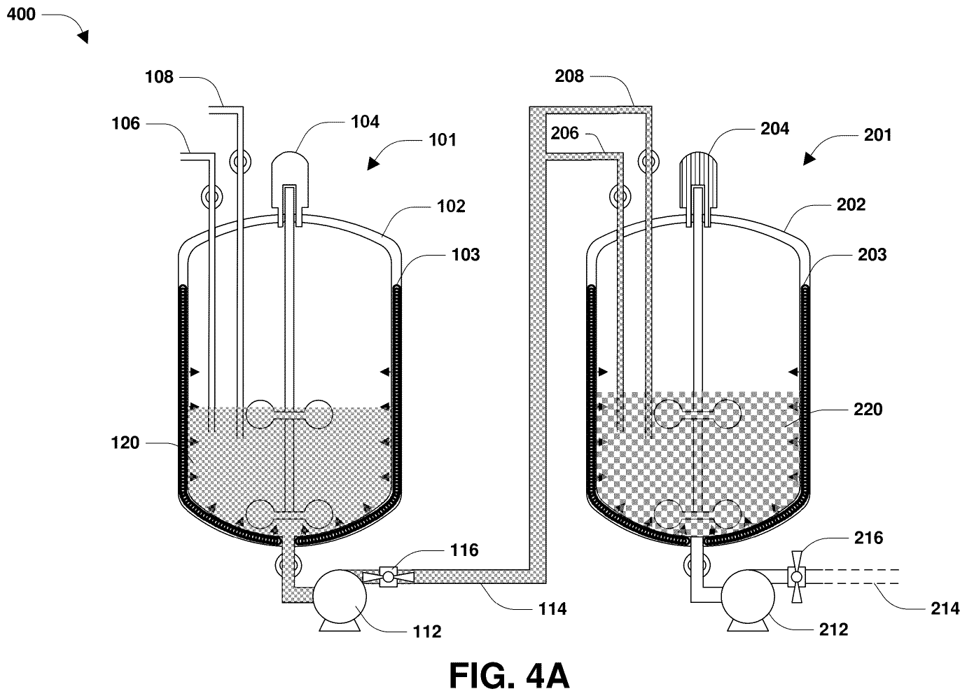

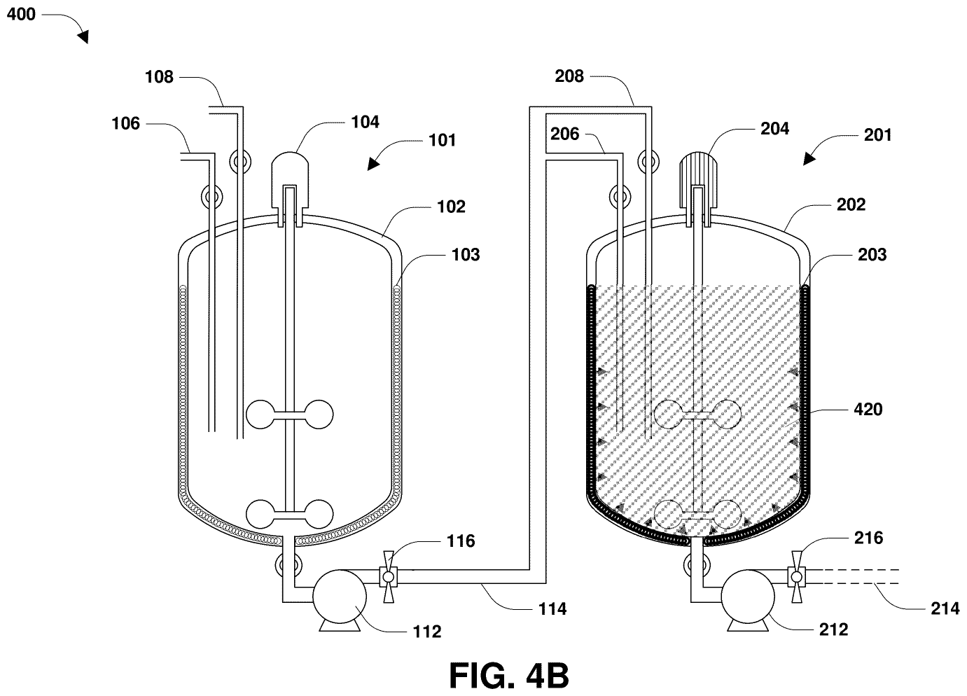

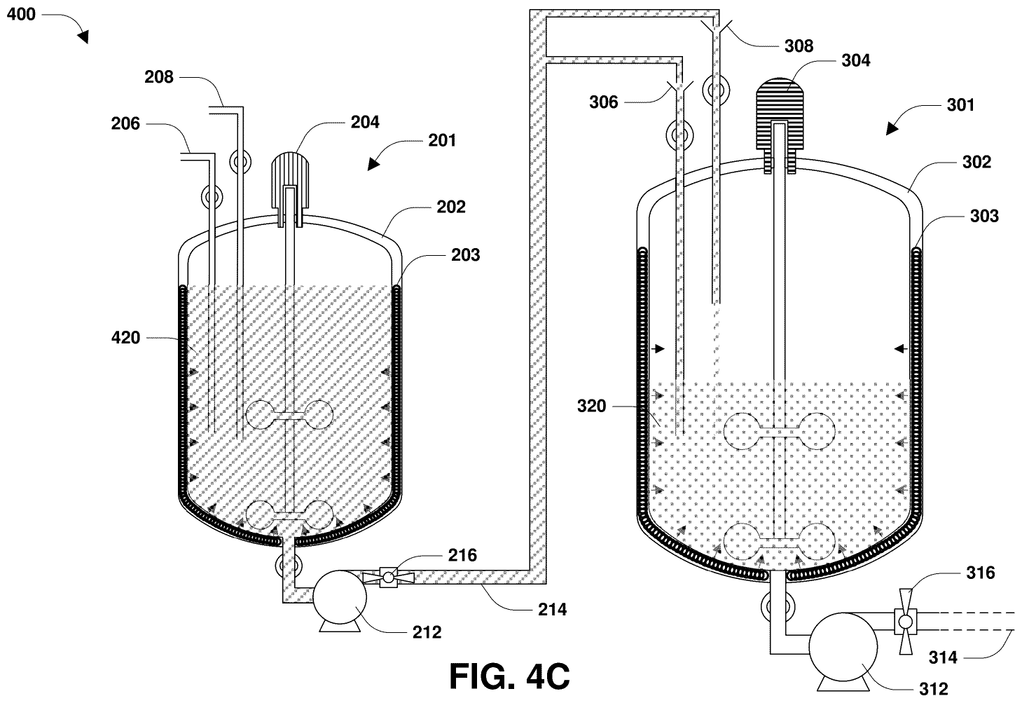

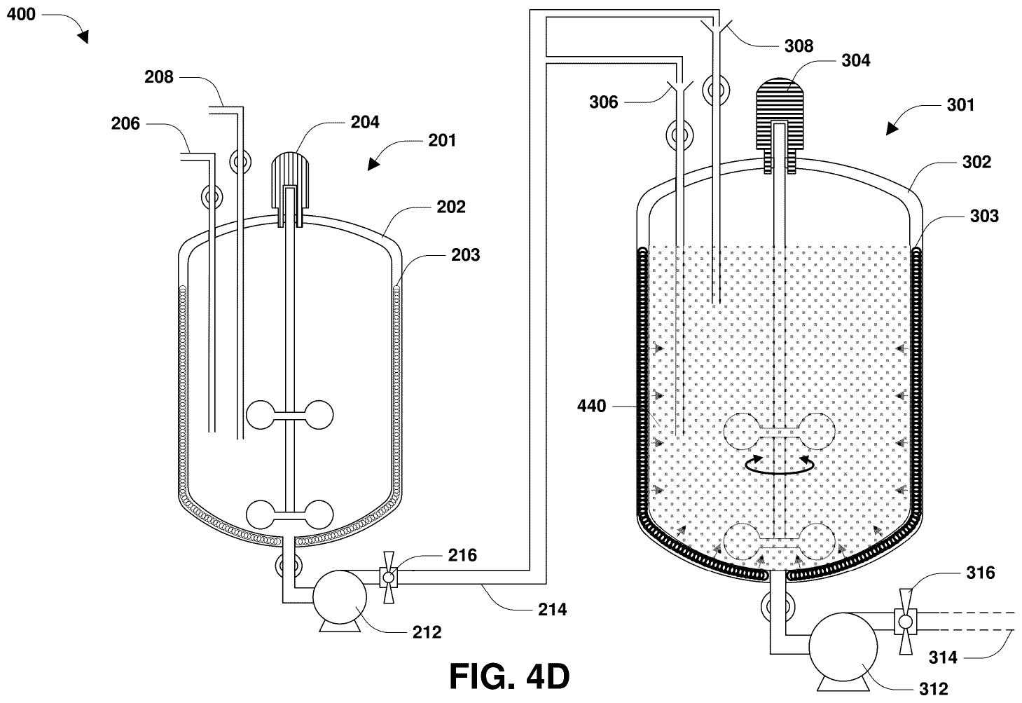

FIG. 4A-4D illustrate an example scenario 400 in which one or more mixtures are combined, in accordance to some embodiments. As shown in FIG. 4A, the first mixture 120 within the first reactor 101 may be transferred to the second reactor 201 via the first pump 112. In some examples, the first valve 116 may be opened to allow the first mixture 120 move through the first outlet 114. In some examples, the first mixture 120 may enter the second chamber 202 via the inlet 206 and/or the inlet 208. In FIG. 4B, the first mixture 120 and the second mixture 220 (shown in FIG. 4A) are combined to form a fourth mixture 420. In some examples, the second mixer 204 is used to form the fourth mixture 420. In some examples, the second mixer 204 may stir the fourth mixture 420 with a fourth stirring speed in range of about 10 rpm to about 250 rpm. In some examples, the second mixer 204 may stir the fourth mixture 420 with the fourth stirring speed in range of about 50 rpm to about 100 rpm. In some examples, the fourth temperature of the second heater 203 may be controlled (using the second heater 203, for example) to be one or more temperatures within a fourth temperature range. In some examples, the fourth temperature range ranges from between about 30 degrees Celsius to about 80 degrees Celsius. In some examples, the fourth temperature range ranges from between about 50 degrees Celsius to about 70 degrees Celsius. In some examples, based upon maintaining the fourth temperate and/or the fourth stirring speed, the fourth mixture 420 is turning into a homogeneous mixture.

In FIG. 4C, the fourth mixture 420 within the second reactor 201 may be transferred to the third reactor 301 via the second pump 212. In some examples, the second valve 216 may be opened to allow the fourth mixture 420 move through the second outlet 214. In some examples, the fourth mixture 420 may enter the third chamber 302 via the inlet 306 and/or the inlet 308. In some examples, the stirring speed of the third mixer 304 may be increased. In FIG. 4D, the fourth mixture 420 and the third mixture 320 (shown in FIG. 4C) are combined to form a fifth mixture 440. In some examples, a third mixer 304 is used to form the fifth mixture 440. In some examples, the third mixer 304 may stir the fifth mixture 440 with a fifth stirring speed in range of about 10 rpm to about 250 rpm. In some examples, the third mixer 304 may stir the fifth mixture 440 with a fourth stirring speed in range of about 50 rpm to about 100 rpm. In some examples, the fifth temperature of the third heater 303 may be controlled (using the third heater 303, for example) to be one or more temperatures within a fifth temperature range. In some examples, the fifth temperature range ranges from between about 50 degrees Celsius to about 180 degrees Celsius. In some examples, the fifth temperature range ranges from between about 65 degrees Celsius to about 100 degrees Celsius. In some examples, the third mixer 304 may continue mixing the fifth mixture 440 at the fifth temperature for a fifth time period. In some examples, the fifth time period may be in a range of about 5 minutes to about 40 minutes. In some examples, the fifth time period may be in a range of about 10 minutes to about 30 minutes. In some examples, based upon maintaining the fifth temperate and/or the fifth stirring speed, the fifth mixture 440 is turning into a homogeneous mixture. In some examples the third heater 303 may decrease the fifth temperature by a range of about 1 degree Celsius to about 10 degrees Celsius. In some examples, the third heater 303 may decrease the fifth temperature by a range of about 2 degrees Celsius to about 8 degrees Celsius.

In some examples, the third pump 312 may allow transferring the fifth mixture 440 utilizing the third valve 316 and/or the third outlet 314. In some examples, a solidifier device is used to convert the fifth mixture 440 into the wax product.

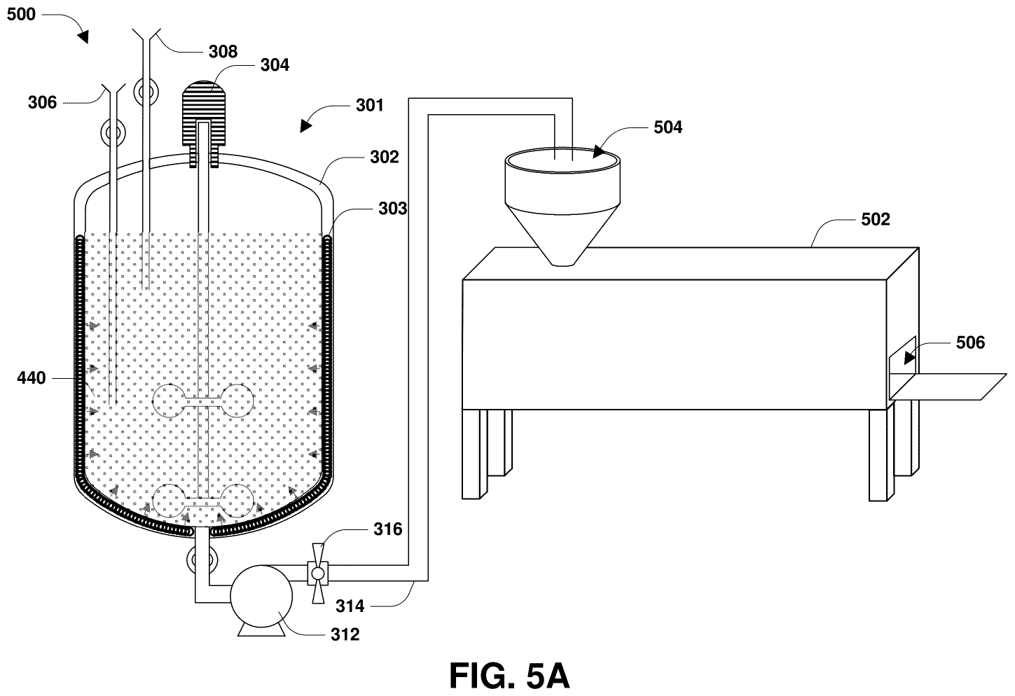

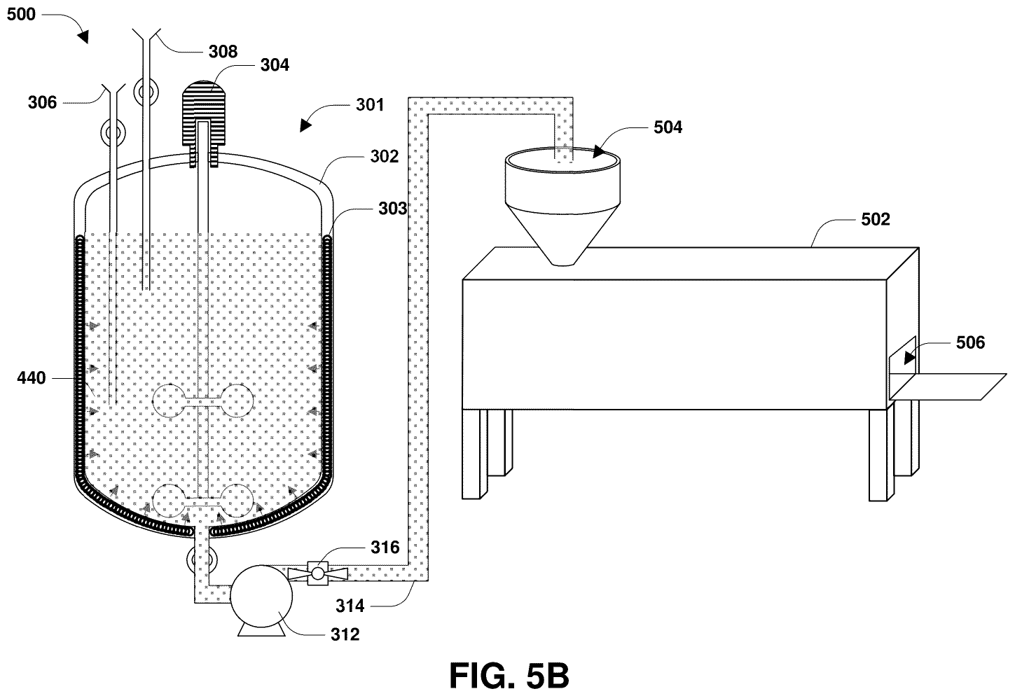

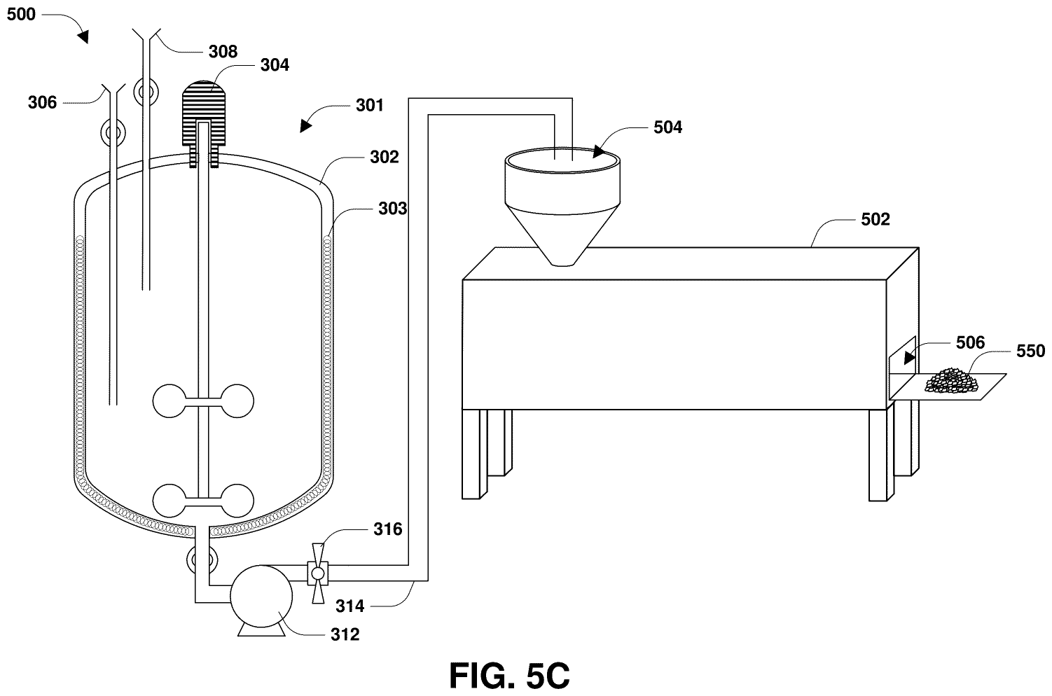

FIG. 5A-5C illustrate an example scenario 500 of producing a wax product 550, in accordance to some embodiments. As show in FIG. 5A, a solidifier device 502 is utilized to produce the wax product 550 (e.g., from the fifth mixture 440). In some examples, the solidifier device 502 may comprise at least one of a pastillator device, or a granulator device. In some examples, the solidifier device 502 may comprise a funnel 504 (e.g., entry, inlet, etc.), a cooling source (not shown) and/or an exit 506. In FIG. 5B, the third pump 312 may allow transferring the fifth mixture 440 utilizing the third valve 316 and/or the third outlet 314. In some examples, the fifth mixture 440 may enter the solidifier device 502 via the funnel 504. In FIG. 5C, based upon using the cooling source within the solidifier device 502, the temperature of the fifth mixture 440 may decrease. In some examples, the solidifier device 502 may convert the fifth mixture 440 into the wax product 550. In some examples, the wax product 550 may comprise a flake shape and/or granule shape. The wax product 550 may exit the solidifier device 502 via the exit 506.

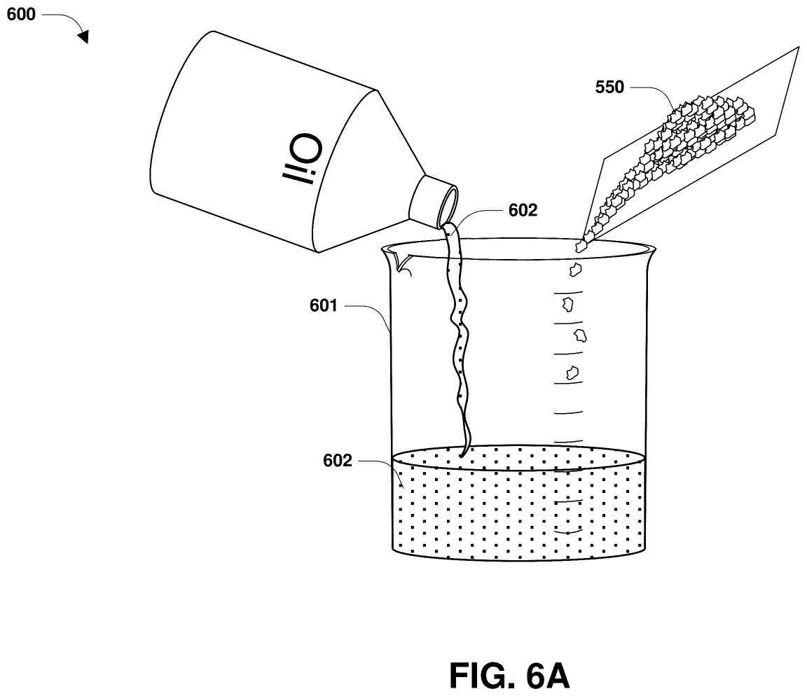

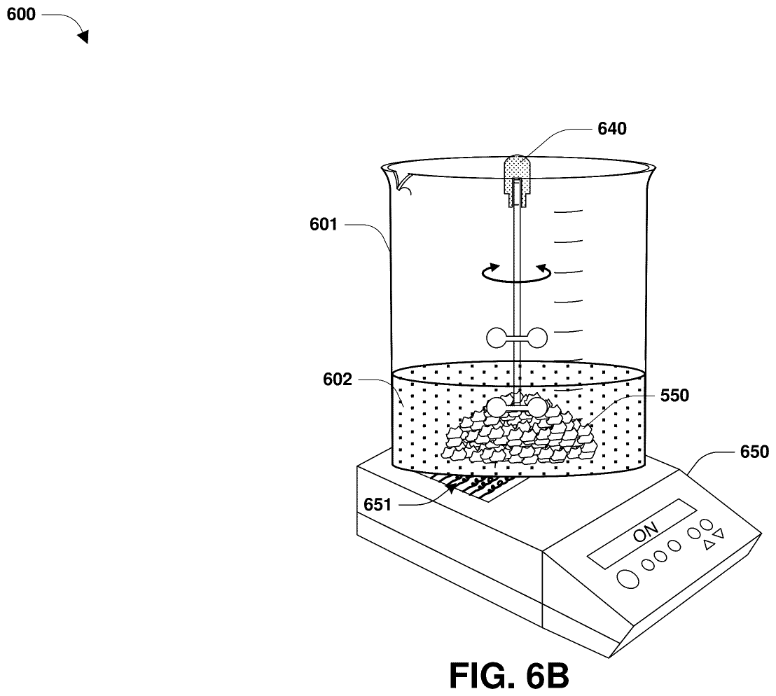

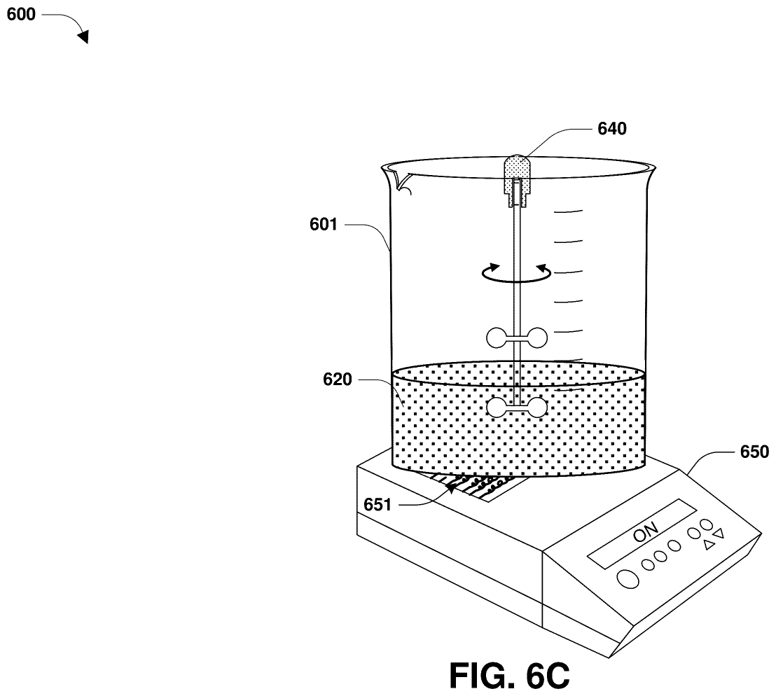

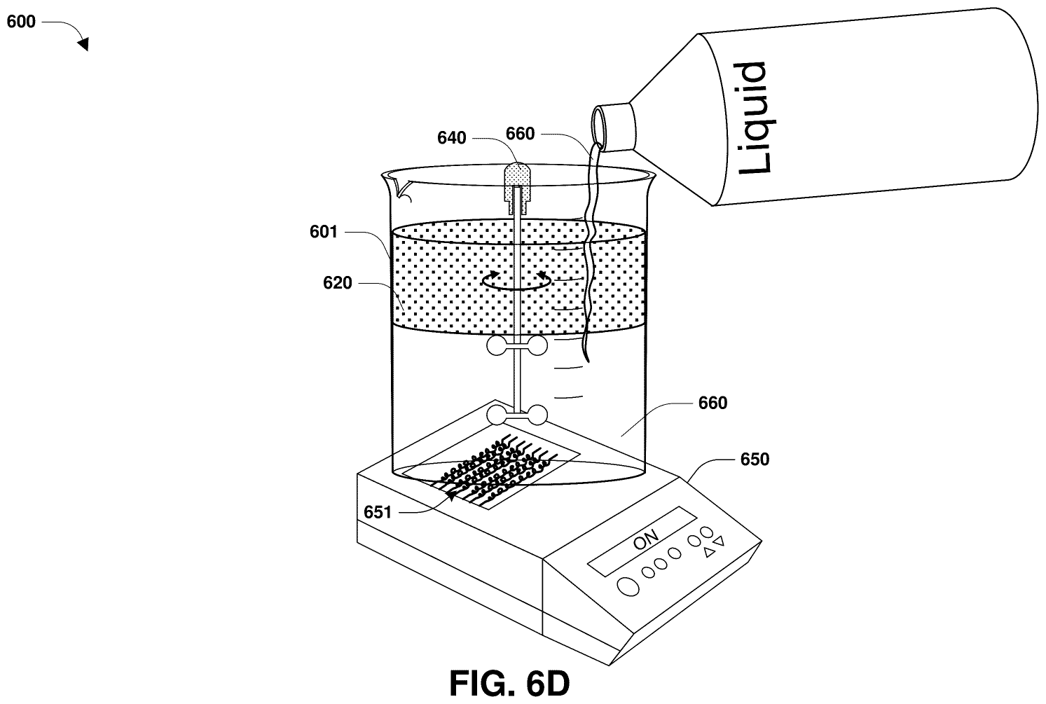

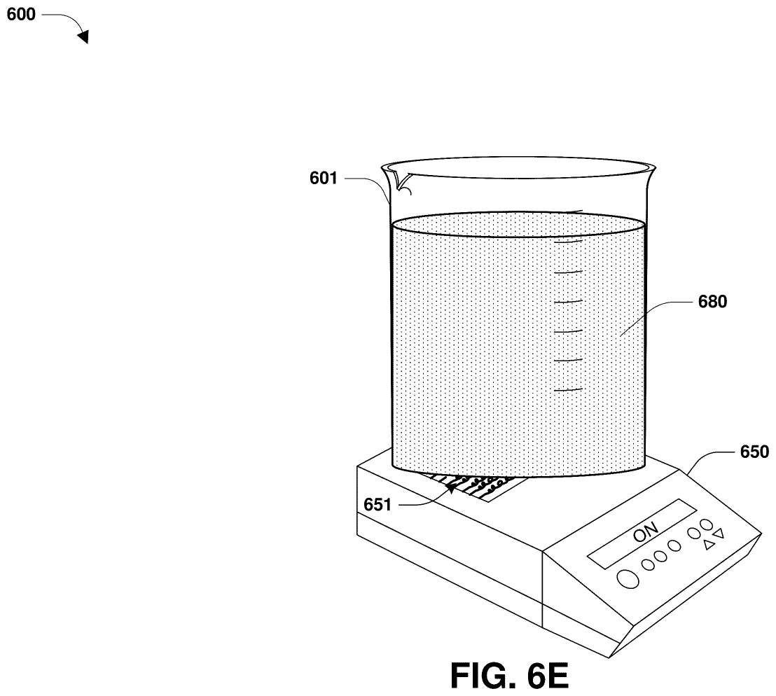

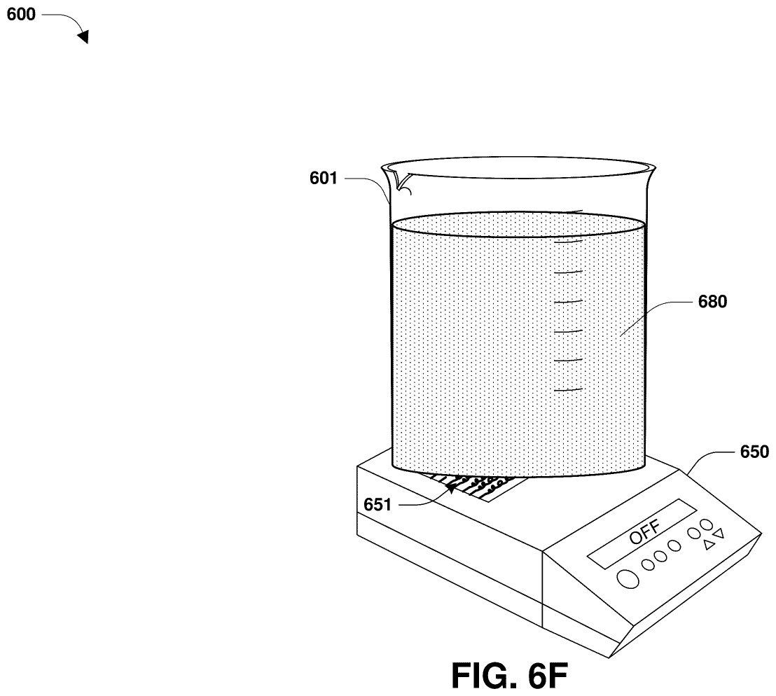

FIG. 6A-6F illustrate an example scenario 600 of making one or more emulsions utilizing the wax product 550, in accordance to some embodiments. As shown in FIG. 6A, the wax product 550 is combined with a first oil 602. In some examples, the wax product 550 and the first oil 602 are poured inside a container 601. In some examples, the first oil 602 may comprise at least one of a mineral oil, a natural oil, or a synthetic oil. In some examples, the container 601 may comprise a beaker, a lab flask, etc.

In FIG. 6B, a mixer 640 (e.g., agitator) is configured to provide a first stirring speed to mix the wax product 550 and/or the first oil 602. In some examples, the first stirring speed is in range of about 10 rpm to about 250 rpm. In some examples, the first stirring speed is in range of about 50 rpm to about 100 rpm. In some examples, a heating device 650 is utilized to heat the wax product 550 and/or the first oil 602. The heating device 650 may comprise a Bunsen burner, a hot plate, a heating mantle, an oven, a furnace, an incubator, and/or a baths (e.g., water bath). In some examples, the heating device 650 may comprise one or more elements 651 to provide a first temperature to the wax product 550 and/or the first oil 602. In some examples, the first temperature may be within the range of about 40 degrees Celsius to about 120 degrees Celsius. In some examples, the first temperature may be within the range of about 60 degrees Celsius to about 100 degrees Celsius (e.g., 85 degrees Celsius).

In FIG. 6C, upon applying the first temperature and/or the first stirring speed, the wax product 550 may dissolve in the first oil 602 and form a first emulsion 620.

In FIG. 6D, the container 601 may be removed (e.g., separated) from the heating device 650. In some examples, a first liquid 660 is added to the first emulsion 620. In some examples, the temperature of the first liquid 660 may be within the range of about 40 degrees Celsius to about 120 degrees Celsius. In some examples, the temperature of the first liquid 660 may be within the range of about 60 degrees Celsius to about 100 degrees Celsius (e.g., 85 degrees Celsius). In some examples, the first liquid 660 may comprise at least one of a deionized water, or a distilled water. In some examples, the mixer 640 maintains the first stirring speed which is in range of about 10 rpm to about 250 rpm.

In FIG. 6E, upon applying the first temperature and/or the first stirring speed, the first liquid 660 may dissolve in the first emulsion 620 and form a second emulsion 680. In some examples, due to addition of the first liquid 660 to the first emulsion 620, the color of the first emulsion 620 may change. In some examples, the temperature of the second emulsion 680 may be decreased to the room temperature (e.g., about 20 degrees Celsius to about 25 degrees Celsius). In some examples, the second emulsion 680 is used to produce at least one of a cosmetic product, a pharmaceutical product. or an industrial product. In some examples, the second emulsion 680 is used to produce at least one of a cream, a lotion, an ointment or a lubricant.

The main advantage of this product is that this material is a multi-purpose material and can be used without any need for other extra materials such as fatty alcohols, emulsifiers, waxes, stabilizers, and gelling agents. it reduces costs and energy of industries and increases the speed of manufacturing health and cosmetic products. The main characters of this material are Fatty alcohols less, Without any emulsifiers, Without any kind of wax, Without any type of stabilizers, without any type of gelling agents.

In some examples, due to mixing one or more mixtures and producing one or more emulsions, the HLB (Hydrophilic-Lipophilic Balance) value of the second emulsion 680 is in a range of a first HLB range. In some examples, the HLB value of the second emulsion 680 may facilitate the dissolvement of liquid (e.g., the first liquid 660) and oil (e.g., the first oil 602) and provide stability for the second emulsion 680.

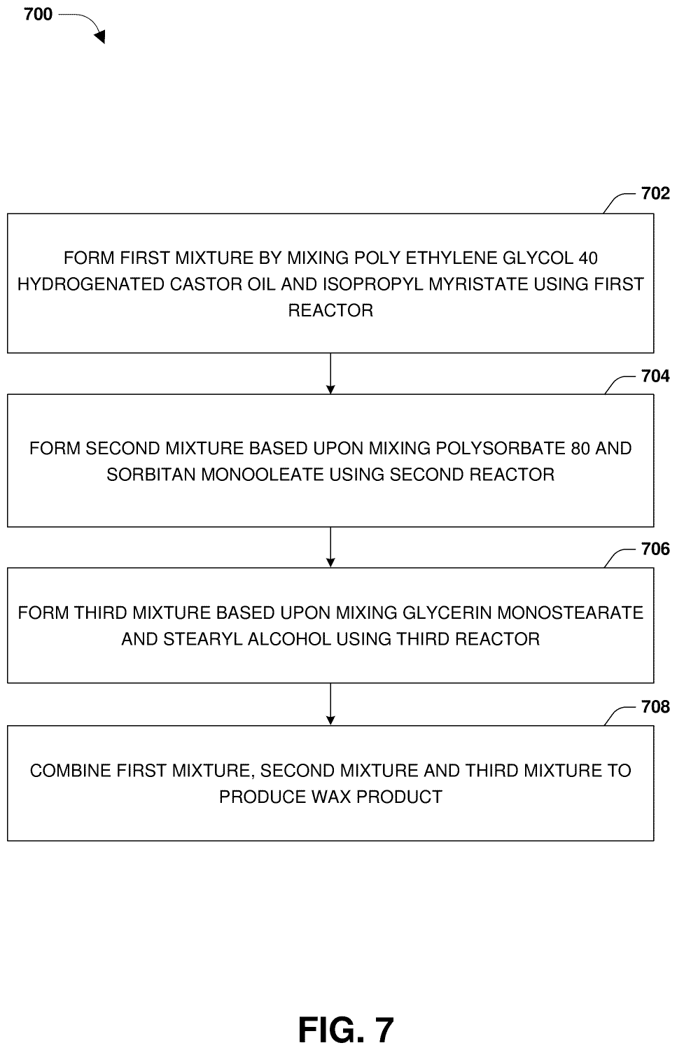

A method 700 for producing a wax product is illustrated in FIG. 7 in accordance to some embodiments. At 702, the method 700 includes forming a first mixture by mixing poly ethylene glycol 40 Hydrogenated Castor Oil (PEG 40 HCO) and Isopropyl myristate (C17H34O2) using a first reactor. At 704, the method 700 includes forming a second mixture based upon mixing Polysorbate 80 (C64H124O26) and Sorbitan monooleate (C24H44O6) using a second reactor. At 706, the method 700 includes forming a third mixture based upon mixing Glycerin monostearate (C21H42O4) and Stearyl Alcohol (C18H38O) using a third reactor. At 708, the method 700 includes combining the first mixture, the second mixture and the third mixture to produce the wax product.

In some examples, the wax product 550 may be used as a surfactant and/or an emulsifier. In some examples, the emulsifier may be a water and oil emulsifier associated with water-in-oil group. In some examples, the wax product 550 may viscosify (e.g., increase viscosity) the one or more products without the addition of other components. In some examples, the wax product 550 may be used to provide a cream. In some examples, the cream may not stick to the skin during and/or after usage. In some examples, the wax product 550 may be used to produce a base cream (e.g., cold cream). In some examples, the base cream may comprise the ability to dissolve water. In some examples, the wax product 550 may not create two phases (e.g., bi phase) during homogenization or mixing. In some examples, the wax product 550 may perform in different PH ranges. In some examples, the wax product 550 may be used as a water and oil solvent without the addition of other substances. In some examples, the one or more products created based upon the wax product 550 may not bi-phase over extended period of time. In some examples, one or more medicinal substances (e.g., health substances) may be carried via the wax product 550. In some examples, the wax product 550 may be used to produce at least one of a skin product, or hair product. In some examples, the wax product 550 may be used to produce one or more emulsions, in which the one or more emulsions may comprise pure white color and/or matte color. In some examples, the wax product 550 may comprise softening properties.

In some examples, a wax product is provided. The wax product includes Polysorbate 80 (C₆₄H₁₂₄O₂₆), Poly ethylene glycol 40 Hydrogenated Castor Oil (PEG 40 HCO), Sorbitan monooleate (C₂₄H₄₄O₆), Isopropyl myristate (C₁₇H₃₄O₂), Glycerin monostearate (C₂₁H₄₂O₄), and Stearyl Alcohol (C₁₈H₃₈O).

In some examples, a ratio of the Polysorbate 80 to the wax product by weight is between about 0.08:1 to about 0.13:1.

In some examples, a ratio of the PEG 40 HCO to the wax product by weight is between about 0.1:1 to about 0.15:1.

In some examples, a ratio of the Sorbitan monooleate to the wax product by weight is between about 0.05:1 to about 0.12:1.

In some examples, a ratio of the Isopropyl myristate to the wax product by weight is between about 0.005:1 to about 0.02:1.

In some examples, a ratio of the Glycerin monostearate to the wax product by weight is between about 0.2:1 to about 0.25:1.

In some examples, a ratio of the Stearyl Alcohol to the wax product by weight is between about 0.3:1 to about 0.4:1.

In some examples, a method for producing a product is provided. The method includes dissolving the wax product in a liquid to form a first emulsion, and dissolving the first emulsion in an oil to form a second emulsion.

In some examples, the second emulsion includes a cosmetic product, a pharmaceutical product, and/or an industrial product.

In some examples, the second emulsion includes a cream, a lotion, an ointment, and/or a lubricant.

In some examples, a ratio of the wax product to the product by weight is between about 0.07:1 to about 0.1:1, and/or between about 0.08:1 to about 0.09:1.

In some examples, a ratio of the liquid to the product by weight is between about 0.6:1 to about 0.8:1, and/or between about 0.7:1 to about 0.75:1.

In some examples, a ratio of the oil to the product by weight is between about 0.1:1 to about 0.3:1, and/or between about 0.15:1 to about 0.25:1.

In some examples, the liquid includes a deionized water, and/or a distilled water.

In some examples, the oil includes a mineral oil, a natural oil, and/or a synthetic oil.

In some examples, a method for producing a wax product includes forming a first mixture by mixing Poly ethylene glycol 40 Hydrogenated Castor Oil (PEG 40 HCO) and Isopropyl myristate (C17H34O2) using a first reactor, forming a second mixture based upon mixing Polysorbate 80 (C64H124O26) and Sorbitan monooleate (C24H44O6) using a second reactor, forming a third mixture based upon mixing Glycerin monostearate (C21H42O4) and Stearyl Alcohol (C18H38O) using a third reactor, and combining the first mixture, the second mixture and the third mixture to produce the wax product.

In some examples, the forming the first mixture includes transferring the PEG 40 HCO into a first reactor chamber defined by the first reactor, wherein a ratio of the PEG 40 HCO to the wax product by weight is between about 0.1:1 to about 0.15:1; increasing a first reactor temperature associated with the first reactor to a temperature within a first temperature range, wherein the first temperature range is at least one of: between about 30 degrees Celsius to about 80 degrees Celsius; or between about 50 degrees Celsius to about 70 degrees Celsius; and while the first reactor temperature is within the first temperature range: transferring the Isopropyl myristate into the first reactor chamber defined by the first reactor, wherein a ratio of the Isopropyl myristate to the wax product by weight is between about 0.005:1 to about 0.02:1; and mixing the PEG 40 HCO and the Isopropyl myristate using a mixer.

In some examples, the forming the second mixture includes transferring the Polysorbate 80 into a second reactor chamber defined by the second reactor, wherein a ratio of the Polysorbate 80 to the wax product by weight is between about 0.08:1 to about 0.13:1; increasing a second reactor temperature associated with the second reactor to a temperature within a second temperature range, wherein the second temperature range is at least one of: between about 30 degrees Celsius to about degrees 80 Celsius; or between about 50 degrees Celsius to about 70 degrees Celsius; and while the second reactor temperature is within the second temperature range: transferring the Sorbitan monooleate into the second reactor chamber defined by the second reactor, wherein a ratio of the Sorbitan monooleate to the wax product by weight is between about 0.05:1 to about 0.12:1; and mixing the Polysorbate 80 and the Sorbitan monooleate using a mixer.

In some examples, the forming the third mixture includes transferring the glycerin monostearate into a third reactor chamber defined by the third reactor, wherein a ratio of the glycerin monostearate to the wax product by weight is between about 0.2:1 to about 0.25:1; transferring the Stearyl Alcohol into the third reactor chamber defined by the third reactor, wherein a ratio of the Stearyl Alcohol to the wax product by weight is between about 0.3:1 to about 0.4:1; and increasing a third reactor temperature associated with the third reactor to a temperature within a third temperature range, wherein the third temperature range is at least one of: between about 50 degrees Celsius to about 180 degrees Celsius; or between about 65 degrees Celsius to about 100 degrees Celsius; and while the third reactor temperature is within the third temperature range: mixing the glycerin monostearate and the Stearyl Alcohol using a mixer.

In some examples, the combining the first mixture, the second mixture and the third mixture to produce the wax product includes forming a fourth mixture by combining the first mixture and the second mixture using a fourth reactor; increasing a fourth reactor temperature associated with the fourth reactor to a temperature within a fourth temperature range, wherein the fourth temperature range is at least one of: between about 30 degrees Celsius to about 80 degrees Celsius; or between about 50 degrees Celsius to about 70 degrees Celsius; and while the fourth reactor temperature is within the fourth temperature range, stirring the fourth mixture for a time period using a mixer, wherein the time period is at least one of between about 1 minutes to about 20 minutes, or between about 5 minutes to about 10 minutes.

In some examples, the combining the first mixture, the second mixture and the third mixture to produce the wax product includes forming a fifth mixture by combining the fourth mixture and the third mixture using a fifth reactor; increasing a fifth reactor temperature associated with the fifth reactor to a temperature within a fifth temperature range, wherein the fifth temperature range is at least one of: between about 50 degrees Celsius to about 180 degrees Celsius; or between about 65 degrees Celsius to about 100 degrees Celsius; while the fifth reactor temperature is within the fifth temperature range, stirring the fifth mixture for a second time period using a mixer, wherein the second time period is at least one of between about 1 minutes to about 20 minutes, or between about 5 minutes to about 10 minutes; in response to performing the stirring of the fifth mixture, decreasing the fifth reactor temperature associated with the fifth reactor by a temperature decrease, wherein the temperature decrease is at least one of: between about 1 degree Celsius to about 10 degrees Celsius; or between about 2 degrees Celsius to about 8 degrees Celsius; and converting the fifth mixture into the wax product.

In some examples, the converting the fifth mixture into the wax product is performed using a solidifier device.

In some examples, the solidifier device includes a pastillator device, and/or a granulator device.

In some examples, a product is provided. The product includes Polysorbate 80 (C₆₄H₁₂₄O₂₆), Poly ethylene glycol 40 Hydrogenated Castor Oil (PEG 40 HCO), Sorbitan monooleate (C₂₄H₄₄O₆), Isopropyl myristate (C₁₇H₃₄O₂), Glycerin monostearate (C₂₁H₄₂O₄), Stearyl Alcohol (C₁₈H₃₈O), liquid, and oil.

In some examples, a ratio of the Polysorbate 80 to the product by weight is between about 0.008:1 to about 0.016:1.

In some examples, a ratio of the PEG 40 HCO to the product by weight is between about 0.01:1 to about 0.02:1.

In some examples, a ratio of the Sorbitan monooleate to the product by weight is between about 0.005:1 to about 0.015:1.

In some examples, a ratio of the Isopropyl myristate to the product by weight is between about 0.0005:1 to about 0.002:1.

In some examples, a ratio of the Glycerin monostearate to the product by weight is between about 0.02:1 to about 0.03:1.

In some examples, a ratio of the Stearyl Alcohol to the product by weight is between about 0.03:1 to about 0.05:1.

In some examples, a ratio of the liquid to the product by weight is between about 0.65:1 to about 0.75:1.

In some examples, a ratio of the oil to the product by weight is between about 0.15:1 to about 0.25:1.

In some examples, the liquid includes a deionized water, and/or a distilled water.

In some examples, the oil includes a mineral oil, a natural oil, and/or a synthetic oil.

Unless specified otherwise, “first,” “second,” and/or the like are not intended to imply a temporal aspect, a spatial aspect, an ordering, etc. Rather, such terms are merely used as identifiers, names, etc. for features, elements, items, etc. For example, a first object and a second object generally correspond to object A and object B or two different or two identical objects or the same object.

Moreover, “example” is used herein to mean serving as an instance, illustration, etc., and not necessarily as advantageous. As used herein, “or” is intended to mean an inclusive “or” rather than an exclusive “or”. In addition, “a” and “an” as used in this application are generally be construed to mean “one or more” unless specified otherwise or clear from context to be directed to a singular form. Also, at least one of A and B and/or the like generally means A or B or both A and B. Furthermore, to the extent that “includes”, “having”, “has”, “with”, and/or variants thereof are used in either the detailed description or the claims, such terms are intended to be inclusive in a manner similar to the term “comprising”.

Although the subject matter has been described in language specific to structural features and/or methodological acts, it is to be understood that the subject matter defined in the appended claims is not necessarily limited to the specific features or acts described above. Rather, the specific features and acts described above are disclosed as example forms of implementing at least some of the claims.

Various operations of embodiments and/or examples are provided herein. The order in which some or all of the operations are described herein should not be construed as to imply that these operations are necessarily order dependent. Alternative ordering will be appreciated by one skilled in the art having the benefit of this description. Further, it will be understood that not all operations are necessarily present in each embodiment and/or example provided herein. Also, it will be understood that not all operations are necessary in some embodiments and/or examples.

Also, although the disclosure has been shown and described with respect to one or more implementations, equivalent alterations and modifications will occur to others skilled in the art based upon a reading and understanding of this specification and the annexed drawings. The disclosure includes all such modifications and alterations and is limited only by the scope of the following claims. In particular regard to the various functions performed by the above described components (e.g., elements, resources, etc.), the terms used to describe such components are intended to correspond, unless otherwise indicated, to any component which performs the specified function of the described component (e.g., that is functionally equivalent), even though not structurally equivalent to the disclosed structure. In addition, while a particular feature of the disclosure may have been disclosed with respect to only one of several implementations, such feature may be combined with one or more other features of the other implementations as may be desired and advantageous for any given or particular application.

What is claimed is:

1. A wax product comprising:
Polysorbate 80 (C₆₄H₁₂₄O₂₆);
Poly ethylene glycol 40 Hydrogenated Castor Oil (PEG 40 HCO);
Sorbitan monooleate (C₂₄H₄₄O₆);
Isopropyl myristate (C₁₇H₃₄O₂);
Glycerin monostearate (C₂₁H₄₂O₄); and
Stearyl Alcohol (C₁₈H₃₈O).

2. The wax product of claim 1, wherein:
a ratio of the Polysorbate 80 to the wax product by weight is between about 0.08:1 to about 0.13:1;
a ratio of the PEG 40 HCO to the wax product by weight is between about 0.1:1 to about 0.15:1;
a ratio of the Sorbitan monooleate to the wax product by weight is between about 0.05:1 to about 0.12:1;
a ratio of the Isopropyl myristate to the wax product by weight is between about 0.005:1 to about 0.02:1;
a ratio of the Glycerin monostearate to the wax product by weight is between about 0.2:1 to about 0.25:1; and
a ratio of the Stearyl Alcohol to the wax product by weight is between about 0.3:1 to about 0.4:1.

3. A method for producing a product comprising:
dissolving the wax product of claim 1 in a liquid to form a first emulsion; and
dissolving the first emulsion in an oil to form a second emulsion.

4. The wax product of claim 3, wherein the second emulsion comprises at least one of:
a cosmetic product;
a pharmaceutical product; or
an industrial product.

5. The wax product of claim 3, wherein the second emulsion comprises at least one of:
a cream;
a lotion;
an ointment; or
a lubricant.

6. The method of claim 3, wherein:
a ratio of the wax product to the product by weight is at least one of:
between about 0.07:1 to about 0.1:1; or
between about 0.08:1 to about 0.09:1;
a ratio of the liquid to the product by weight is at least one of:
between about 0.6:1 to about 0.8:1; or
between about 0.7:1 to about 0.75:1; and
a ratio of the oil to the product by weight is at least one of:
between about 0.1:1 to about 0.3:1; or
between about 0.15:1 to about 0.25:1.

7. The method of claim 3, wherein the liquid comprises at least one of:
a deionized water; or
a distilled water.

8. The method of claim 3, wherein the oil comprises at least one of:
a mineral oil;
a natural oil; or
a synthetic oil.

9. A method for producing a wax product, comprising:
forming a first mixture by mixing Poly ethylene glycol 40 Hydrogenated Castor Oil (PEG 40 HCO) and Isopropyl myristate (C₁₇H₃₄O₂) using a first reactor;
forming a second mixture based upon mixing Polysorbate 80 (C₆₄H₁₂₄O₂₆) and Sorbitan monooleate (C₂₄H₄₄O₆) using a second reactor;
forming a third mixture based upon mixing Glycerin monostearate (C₂₁H₄₂O₄) and Stearyl Alcohol (C₁₈H₃₈O) using a third reactor; and
combining the first mixture, the second mixture and the third mixture to produce the wax product.

10. The method of claim 9, wherein forming the first mixture comprises:
transferring the PEG 40 HCO into a first reactor chamber defined by the first reactor, wherein a ratio of the PEG 40 HCO to the wax product by weight is between about 0.1:1 to about 0.15:1;
increasing a first reactor temperature associated with the first reactor to a temperature within a first temperature range, wherein the first temperature range is at least one of:
between about 30 degrees Celsius to about 80 degrees Celsius; or
between about 50 degrees Celsius to about 70 degrees Celsius; and
while the first reactor temperature is within the first temperature range:
transferring the Isopropyl myristate into the first reactor chamber defined by the first reactor, wherein a ratio of the Isopropyl myristate to the wax product by weight is between about 0.005:1 to about 0.02:1; and
mixing the PEG 40 HCO and the Isopropyl myristate using a mixer.

11. The method of claim 9, wherein forming the second mixture comprises:
transferring the Polysorbate 80 into a second reactor chamber defined by the second reactor, wherein a ratio of the Polysorbate 80 to the wax product by weight is between about 0.08:1 to about 0.13:1;
increasing a second reactor temperature associated with the second reactor to a temperature within a second temperature range, wherein the second temperature range is at least one of:
between about 30 degrees Celsius to about 80 degrees Celsius; or
between about 50 degrees Celsius to about 70 degrees Celsius; and
while the second reactor temperature is within the second temperature range:
transferring the Sorbitan monooleate into the second reactor chamber defined by the second reactor, wherein a ratio of the Sorbitan monooleate to the wax product by weight is between about 0.05:1 to about 0.12:1; and
mixing the Polysorbate 80 and the Sorbitan monooleate using a mixer.

12. The method of claim 9, wherein forming the third mixture comprises:
transferring the glycerin monostearate into a third reactor chamber defined by the third reactor, wherein a ratio of the glycerin monostearate to the wax product by weight is between about 0.2:1 to about 0.25:1;
transferring the Stearyl Alcohol into the third reactor chamber defined by the third reactor, wherein a ratio of the Stearyl Alcohol to the wax product by weight is between about 0.3:1 to about 0.4:1; and
increasing a third reactor temperature associated with the third reactor to a temperature within a third temperature range, wherein the third temperature range is at least one of:
between about 50 degrees Celsius to about 180 degrees Celsius; or
between about 65 degrees Celsius to about 100 degrees Celsius; and
while the third reactor temperature is within the third temperature range:
mixing the glycerin monostearate and the Stearyl Alcohol using a mixer.

13. The method of claim 9, wherein the combining the first mixture, the second mixture and the third mixture to produce the wax product comprises:
forming a fourth mixture by combining the first mixture and the second mixture using a fourth reactor;
increasing a fourth reactor temperature associated with the fourth reactor to a temperature within a fourth temperature range, wherein the fourth temperature range is at least one of:
between about 30 degrees Celsius to about 80 degrees Celsius; or
between about 50 degrees Celsius to about 70 degrees Celsius; and
while the fourth reactor temperature is within the fourth temperature range, stirring the fourth mixture for a time period using a mixer, wherein the time period is at least one of between about 1 minutes to about 20 minutes, or between about 5 minutes to about 10 minutes.

14. The method of claim 13, wherein the combining the first mixture, the second mixture and the third mixture to produce the wax product comprises:
forming a fifth mixture by combining the fourth mixture and the third mixture using a fifth reactor;
increasing a fifth reactor temperature associated with the fifth reactor to a temperature within a fifth temperature range, wherein the fifth temperature range is at least one of:
between about 50 degrees Celsius to about degrees 180 Celsius; or
between about 65 degrees Celsius to about 100 degrees Celsius;
while the fifth reactor temperature is within the fifth temperature range, stirring the fifth mixture for a second time period using a mixer, wherein the second time period is at least one of between about 1 minutes to about 20 minutes, or between about 5 minutes to about 10 minutes;
in response to performing the stirring of the fifth mixture, decreasing the fifth reactor temperature associated with the fifth reactor by a temperature decrease, wherein the temperature decrease is at least one of:
between about 1 degree Celsius to about 10 degrees Celsius; or
between about 2 degrees Celsius to about 8 degrees Celsius; and
converting the fifth mixture into the wax product.

15. The method of claim 14, wherein converting the fifth mixture into the wax product is performed using a solidifier device.

16. The method of claim 15, wherein the solidifier device comprises at least one of:
a pastillator device; or
a granulator device.

17. A product comprising:
Polysorbate 80 (C₆₄H₁₂₄O₂₆);
Poly ethylene glycol 40 Hydrogenated Castor Oil (PEG 40 HCO);
Sorbitan monooleate (C₂₄H₄₄O₆);
Isopropyl myristate (C₁₇H₃₄O₂);
Glycerin monostearate (C₂₁H₄₂O₄);
Stearyl Alcohol (C₁₈H₃₈O);
liquid; and
oil.

18. The product of claim 17, wherein:
a ratio of the Polysorbate 80 to the product by weight is between about 0.008:1 to about 0.016:1;
a ratio of the PEG 40 HCO to the product by weight is between about 0.01:1 to about 0.02:1;
a ratio of the Sorbitan monooleate to the product by weight is between about 0.005:1 to about 0.015:1;
a ratio of the Isopropyl myristate to the product by weight is between about 0.0005:1 to about 0.002:1;
a ratio of the Glycerin monostearate to the product by weight is between about 0.02:1 to about 0.03:1;
a ratio of the Stearyl Alcohol to the product by weight is between about 0.03:1 to about 0.05:1.
a ratio of the liquid to the product by weight is between about 0.65:1 to about 0.75:1; and
a ratio of the oil to the product by weight is between about 0.15:1 to about 0.25:1.

19. The product of claim 17, wherein the liquid comprises at least one of:
a deionized water; or
a distilled water.

20. The product of claim 17, wherein the oil comprises at least one of:
a mineral oil;
a natural oil; or
a synthetic oil.
